(19) United States
(12) Patent Application Publication
(10) Pub. No.: US 2026/0060925 A1
(43) Pub. Date: Mar. 5, 2026

(54) PROCESS FOR THE MANUFACTURING OF MICROSCALE HOLLOW METAL BODIES

(71) Applicant: Max-Planck-Institut für Nachhaltige Materialien Gesellschaft mit beschränkter Haftung, Düsseldorf (DE)

(72) Inventors: Rajaprakash RAMACHANDRAMOORTHY, Düsseldorf (DE); Sung-Gyu KANG, Düsseldorf (DE)

(73) Assignee: Max-Planck-Institut für Nachhaltige Materialien Gesellschaft mit beschränkter Haftung, Düsseldorf (DE)

(21) Appl. No.: 19/120,896

(22) PCT Filed: Oct. 17, 2023
(86) PCT No.: PCT/EP2023/078833

§ 371 Date: Apr. 14, 2025

(30) Foreign Application Priority Data
Oct. 17, 2022 (EP) 22201973.9

(51) Int. Cl. A61K 9/00; B33Y 10/00; B33Y 80/00; C25D 1/00

(52) U.S. Cl. CPC A61K 9/0092; B33Y 10/00; B33Y 80/00; C25D 1/003

(57) ABSTRACT

A hollow body has a bottom, an upper end wall, and side walls extending between the bottom and the upper end wall. The upper end wall and side walls are made of metal. The cavity within the hollow body has a volume in the range between 1 picoliter to 1,000 picoliter. The upper end wall comprises at least one opening. The hollow body is suitable for encapsulating small amounts of liquid, or a small micro-electromechanical system, and may be used for the controlled release of pharmaceutical components.

### CROSS-REFERENCE TO RELATED APPLICATION

This application is a national stage application, filed under 35 U.S.C. § 371, of International Patent Application PCT/EP2023/078833, filed on Oct. 17, 2023, which claims the benefit of European Patent Application EP 22201973.9, filed on Oct. 17, 2022.

### TECHNICAL FIELD

The present disclosure relates to a hollow body made of metals obtained by an additive manufacturing process, a process for the manufacturing of the hollow body by an additive manufacturing process and the use of the hollow body for the storage of liquids in the cavity of the hollow body and the release of liquids from the cavity of the hollow body.

### BACKGROUND

Microfabrication using classic UV lithography is suited only for creating two-dimensional structures with topography (2.5D) on very specific substrates such as silicon, which is intrinsically brittle, and a few thin films such as nickel and aluminum nitride. They also typically lack built-in damage sensing or impact protection mechanisms. As such, there is a critical need to identify micromanufacturing techniques that are capable of fabricating true 3D microarchitectures (size scale-smallest dimension: 200 nm-15 μm). Recently, the advent of two-photon lithography (TPL) has enabled the 3D printing of microscale architectures. TPL uses a pulsed femtosecond laser beam with infra-red (IR) wavelength to define freeform 3D polymer structures. Using TPL, a variety of complex microlattice designs and structures with nanometer resolution have been realized previously, but given that they are still polymer structures, they were inherently weak.

Liquid encapsulation in microscale architecture has a myriad of applications including drug delivery, impact protection, damage sensing, etc. But even after 3-4 decades of silicon based microfabrication, liquid encapsulation remains largely out of reach. Only one previous academic exercise has partially achieved such encapsulation but requires extremely complex experimental steps.

Localized electrodeposition in liquid (LEL) is an additive micromanufacturing technique that uses a hollow atomic force microscope tip with a small orifice immersed in a standard three-cell electrochemical cell. Electrochemical ink with metal ions is fed through the tip and they get reduced locally on the working electrode, i.e. the conductive substrate. The localized electrodeposition based additive manufacturing technologies have the potential to fabricate 3D metal microarchitectures. Yet, owing to the complexity of the manufacturing process, excepting simple microneedles, complex architectures such as microlattices, micropillars, microsprings, etc., have not been realized using these techniques.

The structures obtained by an additive manufacturing process, for example by localized electrodeposition in liquid, show some irregularities that occur because there is no perfect connection between the voxels. These irregularities result in holes. Therefore, it is not possible to fill the state of art hollow structures with liquids, as the liquid will leave the structure through the holes.

### SUMMARY

An object of present disclosure is to provide a process for preparing a hollow body made of metal having a cavity in which liquids can be encapsulated by localized electrodeposition and a hollow body obtained by such process that is suitable to take up liquids.

Subject of present disclosure is a process for preparing a hollow body having a bottom, an upper end wall and side walls extending between the bottom and the upper end wall by an additive manufacturing process in which a metal is deposited from an electrolyte solution containing metal ions on a substrate.

The process is carried out by an additive manufacturing process. In such process the voxels are deposited on a substrate. The deposition may be effected layer by layer. The voxels may be deposited voxel by voxel or multiple voxels are deposited simultaneously. It is possible to obtain a hollow body having side walls that are not permeable for liquids.

A further subject of present disclosure is a hollow body having a bottom and side walls, characterized in that
the side walls are made of metal and,
the cavity within the hollow body has a volume in the range between 1 picoliter to 1,000 picoliter and
that it is obtainable by an substrate additive manufacturing process, by an additive manufacturing process in which a metal is deposited from an electrolyte solution containing metal ions on a substrate.

The hollow body may also comprise an upper end wall that is obtained in the course of the manufacturing process of the hollow body and is made of metal. In such embodiment the sidewalls extend between the bottom and the upper end wall. Preferably the upper end wall comprises at least one opening.

The hollow body is manufactured by an additive manufacturing process, preferably by localized electrodeposition in liquid (LEL). The additive manufacturing process and the LEL are known in the art. may comprise the following steps
a. providing an electrically conducting substrate;
b. providing an electrolyte reservoir having an aperture the reservoir containing an electrolyte solution comprising at least one ionic component; the ionic component capable of being electrodeposited to form the selected material; and an electrode in electrical contact with the electrolyte solution;
c. applying a potential difference between the reservoir electrode and the substrate;
d. bringing the aperture of the electrolyte reservoir sufficiently close to the substrate to establish an electrical current between the reservoir electrode and the substrate, thereby electrodepositing the selected material on the substrate.

The LEL technique has the advantage that multiphase printing is possible-as liquids can be potentially encapsulated within metal cavities and the voxel diameter can be controlled scaled by using pressure changes (without change of tip aperture). LEL is a voxel by voxel deposition method allows freeform printing of complex microscale geometries with submicron resolution. It is possible to obtain microarchitecture such as hollow bodies that are structural error free, have low surface roughness, and are fully dense. It is possible to manufacture hollow bodies of any external shape (simple cylinders to complex 3D architectures such as lattices). The shape may be selected depending on the requirements of the area of application. In most applications the hollow body has a simple geometric form such as a cubic, rectangular-shaped or cylindrical form. Usually, the shape of the hollow body depends on the intended application release of liquids in general, a simple cylindrical shape would suffice. But for example, if multifunctionality of impact protection and damage sensing is required, then in that case the hollow shape would be much more complex such as a microlattice geometry, i.e. an architected design with typically repeating unit cells of the same design (compared to randomly shaped foams for example.

The voxel locations were determined from the computer-aided design based model of metal microarchitectures. In a preferred embodiment the voxels are deposited in such a distance that micro/nano voids are avoided. A distance between each voxel can be chosen from 0.3 μm to 2.0 μm, preferably from 0.5 μm to 1.5 μm, to prevent micro/nano voids between deposited metal voxels. The distance can be controlled by the tip position.

In a preferred embodiment the voxels are deposited in such a manner that the voxels overlap. The voxels can overlap in each direction, for example in the horizontal direction, in another embodiment they overlap in the vertical direction. The overlap between the voxels can vary and may be in the range between 0.12 to 0.2, preferably in the range between 0.15 and 0.18. That means obtained by overlapping the adjacent voxels by a particular distance. For example, if the voxel diameter is 3 microns, the overlap should be around 0.5 microns.

For example, the overlaps can be obtained by overlapping the adjacent voxels by a particular distance based on the tip position. In another embodiment the voxel size is controlled by the printing parameters of voltage and air pressure.

The microlattice architecture provide the impact protection, while the encapsulated liquid (with a fluorescence marker) would provide both added impact protection and damage sensing mechanism, using quick fluorescence microscopy inspection.

According to the process it is possible to prepare micro architectures with dimensions up to 1 mm. The walls may have a thickness from 1 μm to 15 μm, preferably from 2 μm to 10 μm. The cavity inside the hollow body may have a volume from 1 picoliter to 1,000 picoliter, preferably from 10 femtoliter to 100 picoliter. In this cavity liquids and/or MEMS (micro-electromechanical system) may be encapsulated. The liquids may be selected from any kind of pharmaceuticals and pharmaceutical containing liquids.

The hollow body is made of metal. Any metal that can be electrodeposited from an aqueous solution can be used to manufacture metal microarchitectures using the LEL process, such as Cu, Ag, Au, Pd, Pt, Co, Ni, Fe and also rare earth metals such as In.

In a preferred embodiment the bottom of the hollow body has the form of a flat body or disc and is made from a conductive material or coated with a conductive material so that it can be used as the working electrode (or counter electrode) in a LEL process. Examples of conductive materials are metals, conductive polymer material including carbon-fiber-reinforced polymers, a wafer made of glass or any other material coated with conductive materials. In one embodiment the bottom of the hollow body is prepared during the electrodeposition process, i.e. the layers of the bottom are prepared first. In another embodiment, the substrate used in the electrodeposition process will be the bottom of the finished product. Depending on the intended use of the finished hollow body, the substrate may be removed after the end of the process.

In a further embodiment the substrate can be a non-conductive substrate, i.e. an insulative substrate, which comprises conductive prints. The conductive wires are drawn from a surrounding conductive working electrode, for example a graphite electrode. The insulative substrate may be arranged on a conductive substrate which is connected to the working electrode.

The upper end wall covers the cavity of the hollow body and has at least one opening over which encapsulated liquid can be released. It may also be named as a final cap. In case the hollow body comprise more than one opening in the upper end wall, these openings can be distributed over the area of the upper end wall. The upper end wall is usually made of the same material as the side walls extending between the bottom and the upper end wall. The opening can correspond to the opening area of the cavity which may be covered with another material after filling liquid or any other components into the cavity.

In one embodiment the upper end wall is covered with a thin film, preferably with a polymer, for example a biodegradable polymer.

In the process, the upper end wall can be obtained for example that the printing starts from the side wall and printing the adjacent voxels outside first (to produce the minor overhang) and then progressively moving inside the hollow cylinder and printing in a sequential vertical line-by-line manner. In one embodiment at least 2 voxel layers in thickness of the cap can completely seal the cylinder without leaks.

In another embodiment at least one of the sidewalls or the upper end wall or the bottom contains a luminescence marker.

The printing variables that require optimization based on the electrodeposition and geometry choice include deposition voltage, air pressure and the orifice diameter.

The localized electrochemical deposition process requires a supporting electrolyte solution for achieving the conductive path between the working and counter/reference electrode, which forms the three-cell electrochemical setup. Using the voxel by voxel deposition submerged inside this supporting electrolyte liquid (aqueous solution of pH 2 with sulfuric and hydrochloric acid), closed-form geometries such as cylinders or shells when printed will have the supporting conductive electrolyte (SCE) sealed within the void. Specifically for sealing liquids successfully within microscale metallic shells, it's also vital to optimize the voxel overlap between adjacent voxels in the layer of printing and within the layers themselves. This prevents the leakage of the encapsulated liquids through small pores.

During the fabrication process, the overpotential can be varied between −0.49V to −0.51V.

The ionic solution can be fed to the container with a certain pressure which can be varied between 20 mbar to 100 mbar, in particular between 40 mbar and 80 mbar.

Voxel diameter can be calibrated by printing a vertical voxel array with various potential and air pressure. Voxel diameter can also vary with the orifice size, the potential, and the air pressure. For example, with the orifice size of 300 nm, the voxel diameter increases from 1 micron to 5 microns as the pressure increases from 30 mbar to 150 mbar. The voxel diameter also slightly increases with decreasing potential. By printing the vertical voxel array and measuring the diameter via a scanning electron microscope, the person skilled in the art will find the parameters for the proper voxel diameter.

Currently, LEL remains one of the two techniques that have the potential to encapsulate liquids, as the fabrication process needs to be conducted submerged in liquid. The other technique which theoretically has the potential to encapsulate liquids similarly is laser induced photoreduction. But is plagued with issues of very high surface roughness and porosity making it currently not suitable again for encapsulation of liquids within metal micro scale architectures, such as hollow bodies which are microvessels for the liquids.

In a further embodiment the encapsulated liquid may contain a luminescence marker. This enables the damage detection of microscale cracks in the walls of the microstructure. For example, if the structure is fractured by external loading (with a microscale crack), there will be a leak of liquid with the marker that can be detected.

The electrochemical deposition process can be customized to a certain extent (within the electrodeposition voltage window with no hydrogen evolution process) by changing the pH and composition of the supporting electrolyte solution, especially for the deposition of different metals. For example, a copper deposition requires typically a pH of between 2-3 in the SCE, while gold deposition requires an alkaline pH of 12. And the low concentration of nanoparticulates (such as nanoparticles, nanosheets) or biological moieties (that do not won't degrade in the pH conditions in the SCE) which won't influence the deposition process, can be introduced in the SCE and can in turn be sealed within the metal microarchitectures.

As mentioned before the electrolyte solution is selected depending on the metal to be deposited. In case the metal is deposited at a pH below 7, i.e. in an acidic solution, for example a mixture of HCl and H₂SO₄, is used. In case the metal is deposited at a pH above 7, preferably above 10, i.e. in an alkaline solution is used, for example an aqueous solution of NaOH and optional KCl and/or Na₂SO₄.

To circumvent the restriction of sealing only the SCE in the metal microarchitectures, hollow architectures with a microscale opening could be fabricated.

Later after the manufacture of such architectures, other liquids such as liquids with different viscosities or drugs could be dispensed using the same hollow AFM tip, to fill the voids. A major advantage is the ability to sense/monitor the filling of the liquid in the cavity using the force feedback based laser signal detection from the top of the AFM cantilever. The force feedback based cutoff for the liquid filling to avoid overflows will be tuned (by changing the cantilever deflection setpoint) according to the density/viscosity of the liquid (as different liquids will impart different resistances on the tip when the hollow architecture is completely filled and the liquid eventually touches the AFM cantilever). Further, these holes on the microarchitectures could be sealed via local deposition or spin coating of polymers such as PLGA. This will enable the possibility to even have timed release of drugs in human biological conditions, as PLGA will degrade over time and encapsulated drugs will be released.

Another subject of the present disclosure is the use of the hollow body for the controlled release of pharmaceutical components.

### BRIEF DESCRIPTION OF THE DRAWINGS

FIGS. 1 and 1A show a microvessel with a hole in the upper wall end.

FIG. 2 shows a 2D plot (x-y plot) of a view on top of the microvessel.

FIG. 3 shows a 3D plot of the printed microvessel.

FIG. 4 shows a schematic view of the LEL process.

FIG. 5 shows filling of liquid into the cavity of the microvessel.

FIG. 6 shows local sealing of the microvessel.

FIG. 7 shows a cross-section of a microvessel filled with liquid and coated with a polymer.

FIG. 8 shows a cross-section of the microvessel of FIG. 7 showing the release of the liquid after degradation of the polymer.

FIG. 9 shows a microvessel filled with liquid and connected with a polymer substrate.

FIG. 10 shows cross-section of a microvessel with an encapsulated MEMS.

FIG. 11 shows an arrangement of the substrate and working electrode using an insulative substrate.

### DETAILED DESCRIPTION

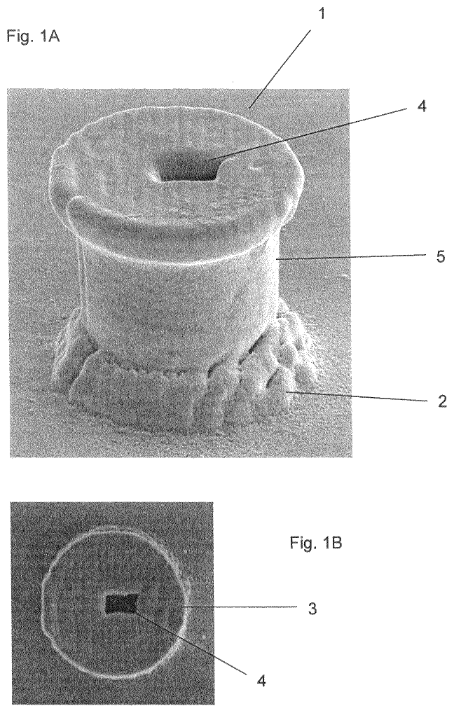

The microvessel shown in FIG. 1 is an example of a hollow body 1. The microvessel has cylindric form, the bottom 2 and the upper end wall 3 are thin discs. The upper end wall 3 comprises a hole 4 through which the liquid can be filled in and through which the liquid can be released into the cavity 5.

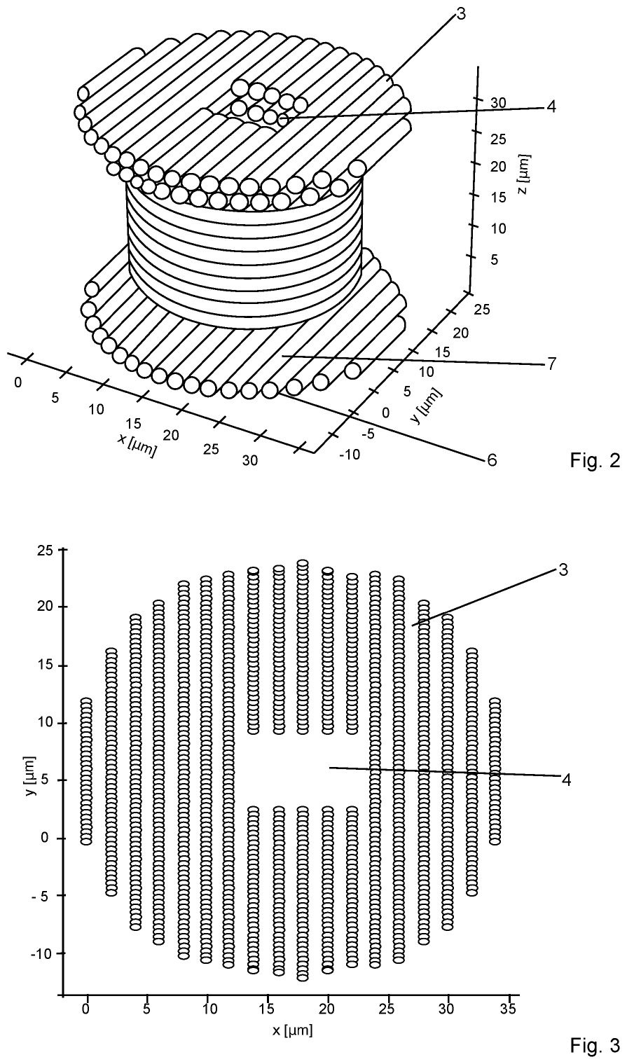

The preferred method for preparing the hollow body 1 is an additive micromanufacturing technique, especially the localized electrodeposition in liquid. A schematic view on top of the microvessel of a x-y plot is shown in FIG. 2 and in FIG. 3 as a 3D plot. As can be seen from the figures, the vessel comprises a bottom 2 consisting of 2 layers 6, 7 ). First, the lower bottom layer 6 is printed and afterwards, the second layer 7 followed by the side walls 8. Finally, the cavity (not shown in this figure) is closed by an upper cover 3. In the embodiment shown in FIGS. 2 and 3 the bottom and the upper end walls are arranged parallel to each other.

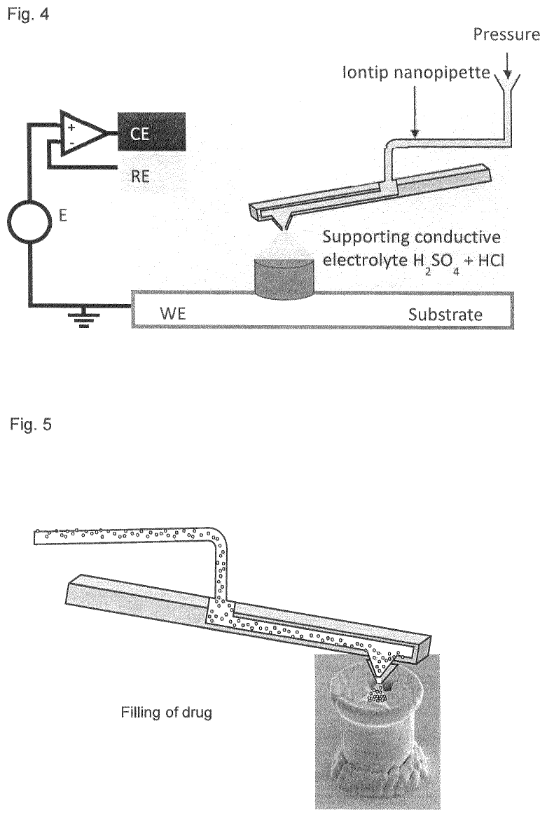

FIG. 4 shows a schematic of the LEL process proceeding. The arrangement consists of an electrolytic cell with a reference electrode, a counter electrode and a working electrode. The voxel grows on the working electrode which is the substrate for the to be printed body. The ionic ink is the starting material for manufacturing the body and is supplied via the reservoir and a nanotip nanopipette to the substrate. The amount of ionic ink and the speed of supply of the ink can be controlled by suitable electronic devices (not shown). Further, the geometry of the body and thickness of the walls can be achieved by adjusting several process parameters such as deposition voltage, air pressure and the orifice diameter.

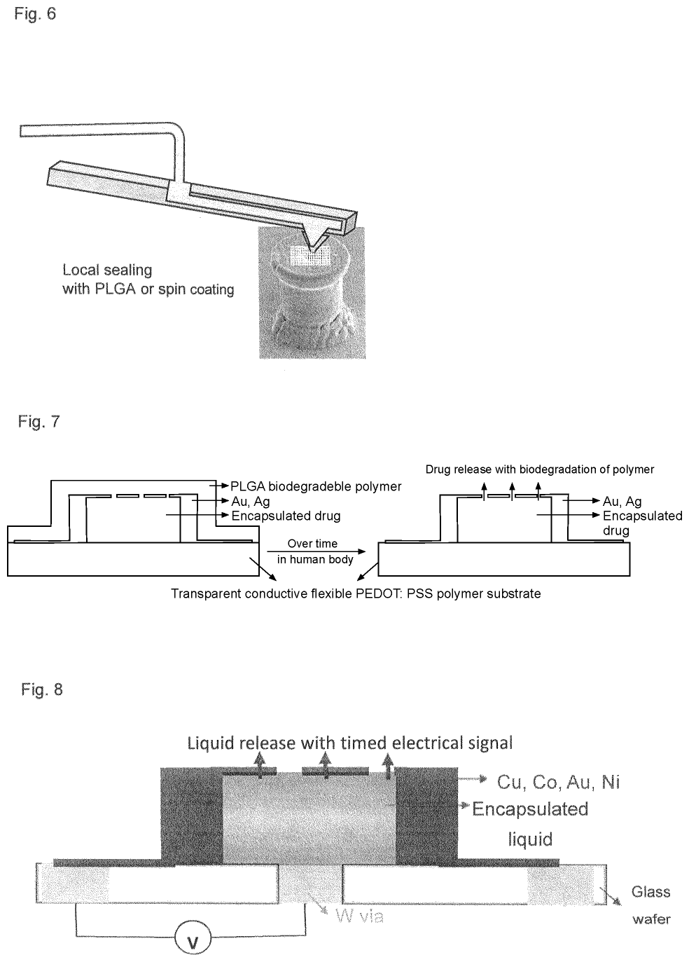

In the next step the obtained microvessel can be filled with a liquid, for example, a drug (s. FIG. 5), afterwards the hole(s) in the upper end wall are closed, for example by local sealing with a polymer, coating area of the hole(s) and around the hole(s) with a polymer, or coating more than only this area by coating the area of the upper end wall whereby the coating may extend also to the side walls (FIG. 6). Preferably, a biodegradable polymer is used for closing or covering the holes, for example polymers on the base of poly lactic acid and polyglycolic acid.

FIG. 7 shows a schematic view of a liquid, for example, a pharmaceutical active agent, encapsulated in a hollow body according to the present disclosure. The side walls and upper end walls are grown voxel by voxel on the working electrode which is the bottom of the final product. The liquid is stored in the cavity of the hollow body. In order to prevent the liquid from leaving the cavity the holes in the upper end wall a closed, i.e. in the embodiment shown in FIG. 7 the upper end wall, the side walls and also part of the bottom and of the substrate are covered with a coating of a biodegradable polymer. When administered as a pharmaceutical agent to a human body, the biodegradable polymer will disintegrate and the closed holes are opened so that the pharmaceutical active is released.

FIG. 8 shows another embodiment. The bottom of the microvessel is a glass wafer with isolated electrical contacts (commercially available as conductive vias on glass substrates) which act as the working electrode in the production process. The release of the encapsulated liquid is initiated by an electrical signal. The voltage source is connected with the encapsulated liquid and the side walls and upper end walls and in case the circuit is closed the liquid will be released.

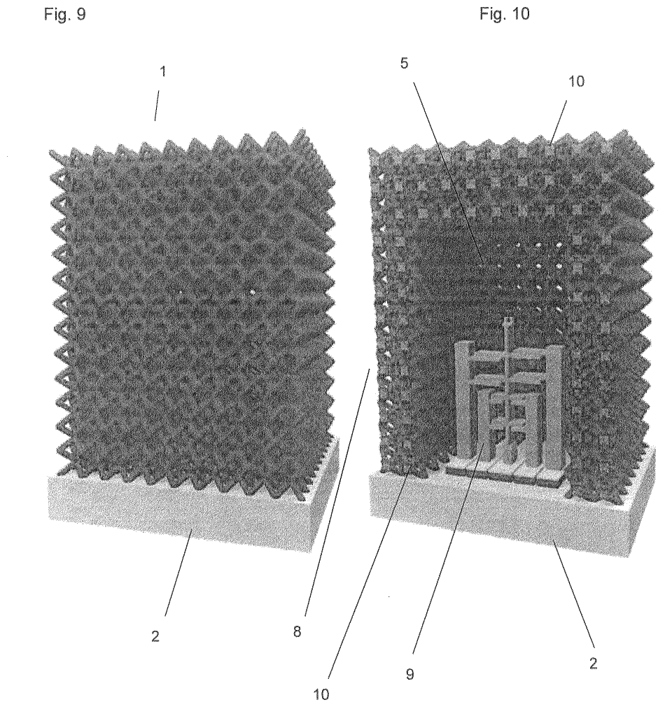

In FIGS. 9 and 10 the hollow body is shown in the form of the lattices of the deposited metal. FIG. 9 shows an external view of the hollow body. FIG. 10 is a sectional view of the hollow body wherein a micro-electromechanical system 9 is arranged in cavity 5 of the hollow body. In this embodiment, the side walls 8 and the upper cover with wall 3 of lattice architecture show a luminescence marker. The walls are the lattices that are hollow and large enough to hold the liquid with a luminescence marker. The marker is located in the lattice of the deposited metal and via this marker any damage to the metal lattice (for example: external particle impacts) can be identified using fluorescence microscopy. In this figure the lattices surrounding the MEMS indeed have porous (as lattices do have low density-which also provides impact protection) unit cells but the struts in the unit cells are themselves hollow encapsulating the liquid with fluorescence dye (required for both enhancing impact protection capability and providing fluorescence based damage sensing when there is an impact/crack etc). In essence, lattices are porous but are themselves hollow with the encapsulating liquid and the lattice structures are the packaging that protects the MEMS in the middle.

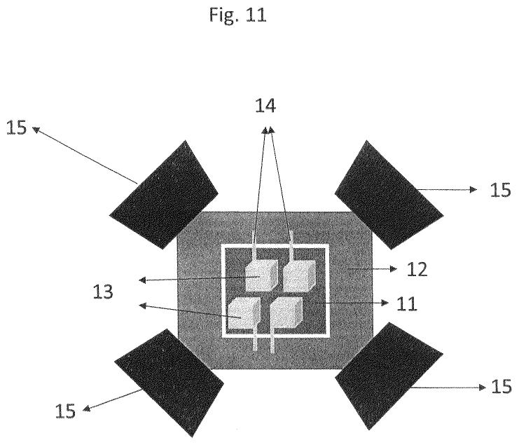

FIG. 11 shows an arrangement for the production of the hollow body using an insulative substrate 11. External graphite electrodes 15 are used as working electrodes. The insulative substrate 11 is applied on a conductive substrate 12. Electrically isolated 3D prints 13 are arranged on the insulative substrate 11 and connected with the working electrode 15 via conductive wires 14.

### EXAMPLE

Liquid containing metal microarchitectures were fabricated via localized electrodeposition in liquid using the CERES system (Exaddon AG, Switzerland). A hollow atomic force microscope tip with a 300-nm-diameter orifice was used. The supporting electrolyte was a solution of 0.5M H₂SO₄ and 0.5 mM HCl. The plating solution was a 0.8M CuSO₄ in 0.5M H₂SO₄ solution.” The process was carried out at room temperature, the applied overpotential was −0.5 V, and the pressure 60 mbar. Voxel locations were determined from the computer-aided design based model of metal microarchitectures. The distance between each voxel was chosen to be 0.5 μm.

As a substrate a 15 mm×15 mm Si-Ti-Cu substrate, which is basically silicon wafer with Ti layer (13 nm) and Cu layer on top (100 nm).

### LIST OF REFERENCE NUMBERS

1 hollow body
2 bottom
3 upper cover wall
4 hole
5 cavity
6 lower layer of the bottom 2
7 second layer of the bottom 2
8 side wall
9 MEMS
10 liquid with fluorescence marker
11 insulative substrate
12 conductive substrate
13 electrically isolated 3D prints
14 conductive wires
15 working electrodes

1.-16. (canceled)

17. An additive manufacturing process, comprising:
preparing a hollow body having
a bottom,
an upper end wall, and side walls extending between the bottom and the upper end wall by depositing a metal from an electrolyte solution containing metal ions on a substrate.

18. The additive manufacturing process according to claim 17,
wherein depositing the metal includes depositing voxels such that they overlap in a horizontal and/or vertical direction.

19. The additive manufacturing process according to claim 18
wherein an overlap between the voxels is between 0.12 to 0.2.

20. The additive manufacturing process according to claim 17, further comprising:
encapsulating a liquid and/or a micro-electromechanical system in the hollow body.

21. The additive manufacturing process according to claim 17, further comprising
encapsulating a liquid pharmaceutical component in the hollow body.

22. A hollow body, having
a bottom,
an upper end wall, and
side walls extending between the bottom and the upper end wall,
wherein the side walls are made of metal, and
wherein a cavity within the hollow body has a volume between 1 picoliter and 1,000 picoliter, and
wherein the hollow body is produced by an additive manufacturing process in which the metal is deposited from an electrolyte solution containing metal ions on a substrate.

23. The hollow body according to claim 22,
wherein the upper end wall is made of the metal.

24. The hollow body according to claim 22,
wherein the upper end wall comprises an opening.

25. The hollow body according to claim 22,
wherein the metal is selected from the group consisting of Cu, Ag, Au, Pd, Pt, Co, Ni, Fe, and rare earth metals.

26. The hollow body according to claim 22,
wherein the bottom is made of metal, a conductive polymer material including carbon-fiber-reinforced polymers, a wafer made of glass, or a material coated with conductive layer.

27. The hollow body according to claim 22,
wherein the upper end wall is covered with a polymer coating.

28. The hollow body according to claim 22,
wherein the hollow body has a cubic, rectangular-shaped, or cylindrical form.

29. The hollow body according to claim 22,
wherein the bottom and the upper end wall are arranged parallel to each other.

30. The hollow body according to claim 22,
wherein at least a part of the side walls and/or the upper end wall comprise a luminescence marker.

31. The hollow body prepared by the additive manufacturing process according to claim 17.

32. A method, comprising:
using the hollow body according to claim 22 for a controlled release of pharmaceutical components.
